SEQUENCE LISTING (20 sequences)

SEQ ID NO: 1 - DNA, 1681 bp, Solanum lycopersicum
atgccgatggataccgaaacaaatcaatcaggagatttcatctttcgatctaagctccctgatatttacatccctaaacatttgcctttacattcttattgctttgaaaatctttcggagttcagttctcgcccctgtttgattgatggttcaaatgatcgaatctacacttatgctgaagttgaacttacttcgagaaaagttgctgttgggcttagcaaattagggatccaacagaaggatacaatcatgatcctcttgcctaattgccctgagtttgtgtttgcgtttattggagcatcgtatctaggagcaatttcaacaatggctaatcctctgtttacaccagcagaggtagtaaaacaagccaaagcctcaagcgctaagatcgttatcacacaagcttgttttgcggggaaagtgaaggactacgcaattgaaaacgatttgaaggttatttgcgtcgactccgcaccggaaggttgtgttcatttctccgaattgattcaatccgatgaacacgaaattcctgaggtgaatatccagccgaacgatgtcgtagctctgccgtattcctccggtactaccgggctgccgaaaggggtgatgttgacacacaaaggcttagtcacgagcgttgcgcaacaagttgatggagaaaatgctaatttgtatatgcacagtgatgatgtgttgatgtgtgtgttgcctctgttccatatatactcactcaattctgttttgctctgtgcattgagagtcggagcagcaattttgattatgcagaaattcgacattgctcagtttttggagttgatacagaagcataaagtgacgattgggccatttgtaccgcctattgttctcgccattgctaagagcccattagttgataactacgatctttcatcagtaagaacagtcatgtctggtgctgcaccattaggcaaggaacttgaagatgccgtccgagccaaattccctaacgctaaactcggtcagggttatggaatgacggaagctggtccagtgctggcgatgtgtttggcatttgcgaaagaaccgtttgatattaaatcaggggcatgtggtaccgttgtgaggaacgcagagatgaaaattgtggatccggatacgggttgctctctgccccgtaaccaacccggtgaaatttgcattagaggtgatcaaatcatgaaaggttacttgaatgaccctgaagctacagctagaacaatagaaaaagaaggatggttacacactggcgatattggatatattgacgatgatgatgagcttttcatcgtggatcgattgaaggaattgatcaaatacaaaggatttcaagtggcgcctgctgaactcgaagcacttcttgtcaaccaccctaacatttctgatgctgctgttgtcccaatgaaagatgaacaagcgggagaagttccagtggcttttgttgttagatcaaatggatctaccattactgaggatgaagtgaaggatttcatctccaagcaggtgatattctataagagaataaagcgtgtatttttcgtggagacggtaccgaaatctccatcaggaaaaattctgagaaaagacttaagagctagactggctgctggtatttcaaattgactcgagacaggccccttttcctttgtcgatatcatgtaattag

SEQ ID NO: 2 - protein, 559 aa, Solanum lycopersicum
MPMDTETNQSGDFIFRSKLPDIYIPKHLPLHSYCFENLSEFSSRPCLIDGSNDRIYTYAEVELTSRKVAVGLSKLGIQQKDTIMILLPNCPEFVFAFIGASYLGAISTMANPLFTPAEVVKQAKASSAKIVITQACFAGKVKDYAIENDLKVICVDSAPEGCVHFSELIQSDEHEIPEVNIQPNDVVALPYSSGTTGLPKGVMLTHKGLVTSVAQQVDGENANLYMHSDDVLMCVLPLFHIYSLNSVLLCALRVGAAILIMQKFDIAQFLELIQKHKVTIGPFVPPIVLAIAKSPLVDNYDLSSVRTVMSGAAPLGKELEDAVRAKFPNAKLGQGYGMTEAGPVLAMCLAFAKEPFDIKSGACGTVVRNAEMKIVDPDTGCSLPRNQPGEICIRGDQIMKGYLNDPEATARTIEKEGWLHTGDIGYIDDDDELFIVDRLKELIKYKGFQVAPAELEALLVNHPNISDAAVVPMKDEQAGEVPVAFVVRSNGSTITEDEVKDFISKQVIFYKRIKRVFFVETVPKSPSGKILRKDLRARLAAGISNCSRQAPFPLSISCN

SEQ ID NO: 3 - DNA, 1170 bp, Solanum lycopersicum
atggtcaccgtggaggagtatcgtaaggcgcaacgtgcagagggtccagctacgatcttagccattggaacatctacgccttctaactgtgttgatcagagtacttatcctgattattattttcgtatcactaacagtgagcacaagactgagctgaaagagaaatttaagcgcatgtgtgataaatcaatgattaagaagagatatatgcacttaaccgaagaaatcttgaaagagaaccctaacatgtgtgcatacatggcaccttcccttgatgcaaggcaagacatagttgttgttgaagtgcctaaacttggaaaaagaggcacccaaaaggccatcaaagaatggggccagcccaaatccaagattacccatttggtcttttgtaccactagtggtgtggacatgcccgcgtgtgactaccaactcgctaagctcctacccgttcgcccatcagtcaagcgactcatgatgtaccaacaaggttgctttgccgggggaacagtacttcggctagccaaggacttggctgagaacaacaagggtgctagagtccttgttgtttgctctgagatcactgcagttacgttccgtggacccagtgaatctcacttagatagcttggtaggccaagccctttttggtgatggggcggccgcaattattataggttcggacccaattataggggtcgaaagacctttatttgaactcgtctcagcagcccaaactcttgtccccgatagcgaaggcgctattgacggacacctacgtgaggttgggcttacattccacttactcaaggatgttcctgggcttatctcgaaaaacatcgaaaagagccttctagaagcatttcaacctctagggatatctgactggaactctctattttggatcgctcaccctggcgggcctgcgattttggaccaagttgaattgaagttgggcctaaagcctgagaaacttagggctacaagagaagtactaagtaactatggcaatatgtcaagtgcttgtgtgttgtttattttggatgaaatgagaaaggcctctacaaaagaaggcctaggaactactggtgaagggcttgaatggggtgtcctttttggatttgggcctgggcttacagttgagactgttgtccttcacagtgttgctgcttag

SEQ ID NO: 4 - protein, 389 aa, Solanum lycopersicum
MVTVEEYRKAQRAEGPATILAIGTSTPSNCVDQSTYPDYYFRITNSEHKTELKEKFKRMCDKSMIKKRYMHLTEEILKENPNMCAYMAPSLDARQDIVVVEVPKLGKRGTQKAIKEWGQPKSKITHLVFCTTSGVDMPACDYQLAKLLPVRPSVKRLMMYQQGCFAGGTVLRLAKDLAENNKGARVLVVCSEITAVTFRGPSESHLDSLVGQALFGDGAAAIIIGSDPIIGVERPLFELVSAAQTLVPDSEGAIDGHLREVGLTFHLLKDVPGLISKNIEKSLLEAFQPLGISDWNSLFWIAHPGGPAILDQVELKLGLKPEKLRATREVLSNYGNMSSACVLFILDEMRKASTKEGLGTTGEGLEWGVLFGFGPGLTVETVVLHSVAA

SEQ ID NO: 5 - DNA, 1681 bp, Solanum lycopersicum
  mutation (703)..(705): mutation of SEQ ID NO1 Val235Met
atgccgatggataccgaaacaaatcaatcaggagatttcatctttcgatctaagctccctgatatttacatccctaaacatttgcctttacattcttattgctttgaaaatctttcggagttcagttctcgcccctgtttgattgatggttcaaatgatcgaatctacacttatgctgaagttgaacttacttcgagaaaagttgctgttgggcttagcaaattagggatccaacagaaggatacaatcatgatcctcttgcctaattgccctgagtttgtgtttgcgtttattggagcatcgtatctaggagcaatttcaacaatggctaatcctctgtttacaccagcagaggtagtaaaacaagccaaagcctcaagcgctaagatcgttatcacacaagcttgttttgcggggaaagtgaaggactacgcaattgaaaacgatttgaaggttatttgcgtcgactccgcaccggaaggttgtgttcatttctccgaattgattcaatccgatgaacacgaaattcctgaggtgaatatccagccgaacgatgtcgtagctctgccgtattcctccggtactaccgggctgccgaaaggggtgatgttgacacacaaaggcttagtcacgagcgttgcgcaacaagttgatggagaaaatgctaatttgtatatgcacagtgatgatgtgttgatgtgtatgttgcctctgttccatatatactcactcaattctgttttgctctgtgcattgagagtcggagcagcaattttgattatgcagaaattcgacattgctcagtttttggagttgatacagaagcataaagtgacgattgggccatttgtaccgcctattgttctcgccattgctaagagcccattagttgataactacgatctttcatcagtaagaacagtcatgtctggtgctgcaccattaggcaaggaacttgaagatgccgtccgaggcaaattccctaacgctaaactcggtcagggttatggaatgacggaagctggtccagtgctggcgatgtgtttggcatttgcgaaagaaccgtttgatattaaatcaggggcatgtggtaccgttgtgaggaacgcagagatgaaaattgtggatccggatacgggttgctctctgccccgtaaccaacccggtgaaatttgcattagaggtgatcaaatcatgaaaggttacttgaatgaccctgaagctacagctagaacaatagaaaaagaaggatggttacacactggcgatattggatatattgacgatgatgatgagcttttcatcgtggatcgattgaaggaattgatcaaatacaaaggatttcaagtggcgcctgctgaactcgaagcacttcttgtcaaccaccctaacatttctgatgctgctgttgtcccaatgaaagatgaacaagcgggagaagttccagtggcttttgttgttagatcaaatggatctaccattactgaggatgaagtgaaggatttcatctccaagcaggtgatattctataagagaataaagcgtgtatttttcgtggagacggtaccgaaatctccatcaggaaaaattctgagaaaagacttaagagctagactggctgctggtatttcaaattgactcgagacaggccccttttcctttgtcgatatcatgtaattag

SEQ ID NO: 6 - protein, 559 aa, Solanum lycopersicum
  VARIANT (235)..(235): variant of SEQ ID NO2 Val235Met
MPMDTETNQSGDFIFRSKLPDIYIPKHLPLHSYCFENLSEFSSRPCLIDGSNDRIYTYAEVELTSRKVAVGLSKLGIQQKDTIMILLPNCPEFVFAFIGASYLGAISTMANPLFTPAEVVKQAKASSAKIVITQACFAGKVKDYAIENDLKVICVDSAPEGCVHFSELIQSDEHEIPEVNIQPNDVVALPYSSGTTGLPKGVMLTHKGLVTSVAQQVDGENANLYMHSDDVLMCMLPLFHIYSLNSVLLCALRVGAAILIMQKFDIAQFLELIQKHKVTIGPFVPPIVLAIAKSPLVDNYDLSSVRTVMSGAAPLGKELEDAVRGKFPNAKLGQGYGMTEAGPVLAMCLAFAKEPFDIKSGACGTVVRNAEMKIVDPDTGCSLPRNQPGEICIRGDQIMKGYLNDPEATARTIEKEGWLHTGDIGYIDDDDELFIVDRLKELIKYKGFQVAPAELEALLVNHPNISDAAVVPMKDEQAGEVPVAFVVRSNGSTITEDEVKDFISKQVIFYKRIKRVFFVETVPKSPSGKILRKDLRARLAAGISNCSRQAPFPLSISCN

SEQ ID NO: 7 - DNA, 1681 bp, Solanum lycopersicum
  mutation (556)..(558): mutation of SEQ ID NO1 Val186Gly
atgccgatggataccgaaacaaatcaatcaggagatttcatctttcgatctaagctccctgatatttacatccctaaacatttgcctttacattcttattgctttgaaaatctttcggagttcagttctcgcccctgtttgattgatggttcaaatgatcgaatctacacttatgctgaagttgaacttacttcgagaaaagttgctgttgggcttagcaaattagggatccaacagaaggatacaatcatgatcctcttgcctaattgccctgagtttgtgtttgcgtttattggagcatcgtatctaggagcaatttcaacaatggctaatcctctgtttacaccagcagaggtagtaaaacaagccaaagcctcaagcgctaagatcgttatcacacaagcttgttttgcggggaaagtgaaggactacgcaattgaaaacgatttgaaggttatttgcgtcgactccgcaccggaaggttgtgttcatttctccgaattgattcaatccgatgaacacgaaattcctgaggtgaatatccagccgaacgatggcgtagctctgccgtattcctccggtactaccgggctgccgaaaggggtgatgttgacacacaaaggcttagtcacgagcgttgcgcaacaagttgatggagaaaatgctaatttgtatatgcacagtgatgatgtgttgatgtgtgtgttgcctctgttccatatatactcactcaattctgttttgctctgtgcattgagagtcggagcagcaattttgattatgcagaaattcgacattgctcagtttttggagttgatacagaagcataaagtgacgattgggccatttgtaccgcctattgttctcgccattgctaagagcccattagttgataactacgatctttcatcagtaagaacagtcatgtctggtgctgcaccattaggcaaggaacttgaagatgccgtccgagccaaattccctaacgctaaactcggtcagggttatggaatgacggaagctggtccagtgctggcgatgtgtttggcatttgcgaaagaaccgtttgatattaaatcaggggcatgtggtaccgttgtgaggaacgcagagatgaaaattgtggatccggatacgggttgctctctgccccgtaaccaacccggtgaaatttgcattagaggtgatcaaatcatgaaaggttacttgaatgaccctgaagctacagctagaacaatagaaaaagaaggatggttacacactggcgatattggatatattgacgatgatgatgagcttttcatcgtggatcgattgaaggaattgatcaaatacaaaggatttcaagtggcgcctgctgaactcgaagcacttcttgtcaaccaccctaacatttctgatgctgctgttgtcccaatgaaagatgaacaagcgggagaagttccagtggcttttgttgttagatcaaatggatctaccattactgaggatgaagtgaaggatttcatctccaagcaggtgatattctataagagaataaagcgtgtatttttcgtggagacggtaccgaaatctccatcaggaaaaattctgagaaaagacttaagagctagactggctgctggtatttcaaattgactcgagacaggccccttttcctttgtcgatatcatgtaattag

SEQ ID NO: 8 - protein, 559 aa, Solanum lycopersicum
  VARIANT (186)..(186): variant of SEQ ID NO2 Val186Gly
MPMDTETNQSGDFIFRSKLPDIYIPKHLPLHSYCFENLSEFSSRPCLIDGSNDRIYTYAEVELTSRKVAVGLSKLGIQQKDTIMILLPNCPEFVFAFIGASYLGAISTMANPLFTPAEVVKQAKASSAKIVITQACFAGKVKDYAIENDLKVICVDSAPEGCVHFSELIQSDEHEIPEVNIQPNDGVALPYSSGTTGLPKGVMLTHKGLVTSVAQQVDGENANLYMHSDDVLMCVLPLFHIYSLNSVLLCALRVGAAILIMQKFDIAQFLELIQKHKVTIGPFVPPIVLAIAKSPLVDNYDLSSVRTVMSGAAPLGKELEDAVRAKFPNAKLGQGYGMTEAGPVLAMCLAFAKEPFDIKSGACGTVVRNAEMKIVDPDTGCSLPRNQPGEICIRGDQIMKGYLNDPEATARTIEKEGWLHTGDIGYIDDDDELFIVDRLKELIKYKGFQVAPAELEALLVNHPNISDAAVVPMKDEQAGEVPVAFVVRSNGSTITEDEVKDFISKQVIFYKRIKRVFFVETVPKSPSGKILRKDLRARLAAGISNCSRQAPFPLSISCN

SEQ ID NO: 9 - DNA, 1681 bp, Solanum lycopersicum
  mutation (715)..(717): mutation of SEQ ID NO1 Phe239Ser
atgccgatggataccgaaacaaatcaatcaggagatttcatctttcgatctaagctccctgatatttacatccctaaacatttgcctttacattcttattgctttgaaaatctttcggagttcagttctcgcccctgtttgattgatggttcaaatgatcgaatctacacttatgctgaagttgaacttacttcgagaaaagttgctgttgggcttagcaaattagggatccaacagaaggatacaatcatgatcctcttgcctaattgccctgagtttgtgtttgcgtttattggagcatcgtatctaggagcaatttcaacaatggctaatcctctgtttacaccagcagaggtagtaaaacaagccaaagcctcaagcgctaagatcgttatcacacaagcttgttttgcggggaaagtgaaggactacgcaattgaaaacgatttgaaggttatttgcgtcgactccgcaccggaaggttgtgttcatttctccgaattgattcaatccgatgaacacgaaattcctgaggtgaatatccagccgaacgatgtcgtagctctgccgtattcctccggtactaccgggctgccgaaaggggtgatgttgacacacaaaggcttagtcacgagcgttgcgcaacaagttgatggagaaaatgctaatttgtatatgcacagtgatgatgtgttgatgtgtgtgttgcctctgtcccatatatactcactcaattctgttttgctctgtgcattgagagtcggagcagcaattttgattatgcagaaattcgacattgctcagtttttggagttgatacagaagcataaagtgacgattgggccatttgtaccgcctattgttctcgccattgctaagagcccattagttgataactacgatctttcatcagtaagaacagtcatgtctggtgctgcaccattaggcaaggaacttgaagatgccgtccgagccaaattccctaacgctaaactcggtcagggttatggaatgacggaagctggtccagtgctggcgatgtgtttggcatttgcgaaagaaccgtttgatattaaatcaggggcatgtggtaccgttgtgaggaacgcagagatgaaaattgtggatccggatacgggttgctctctgccccgtaaccaacccggtgaaatttgcattagaggtgatcaaatcatgaaaggttacttgaatgaccctgaagctacagctagaacaatagaaaaagaaggatggttacacactggcgatattggatatattgacgatgatgatgagcttttcatcgtggatcgattgaaggaattgatcaaatacaaaggatttcaagtggcgcctgctgaactcgaagcacttcttgtcaaccaccctaacatttctgatgctgctgttgtcccaatgaaagatgaacaagcgggagaagttccagtggcttttgttgttagatcaaatggatctaccattactgaggatgaagtgaaggatttcatctccaagcaggtgatattctataagagaataaagcgtgtatttttcgtggagacggtaccgaaatctccatcaggaaaaattctgagaaaagacttaagagctagactggctgctggtatttcaaattgactcgagacaggccccttttcctttgtcgatatcatgtaattag

SEQ ID NO: 10 - protein, 559 aa, Solanum lycopersicum
  VARIANT (239)..(239): variant of SEQ ID NO2 Phe239Ser
MPMDTETNQSGDFIFRSKLPDIYIPKHLPLHSYCFENLSEFSSRPCLIDGSNDRIYTYAEVELTSRKVAVGLSKLGIQQKDTIMILLPNCPEFVFAFIGASYLGAISTMANPLFTPAEVVKQAKASSAKIVITQACFAGKVKDYAIENDLKVICVDSAPEGCVHFSELIQSDEHEIPEVNIQPNDVVALPYSSGTTGLPKGVMLTHKGLVTSVAQQVDGENANLYMHSDDVLMCVLPLSHIYSLNSVLLCALRVGAAILIMQKFDIAQFLELIQKHKVTIGPFVPPIVLAIAKSPLVDNYDLSSVRTVMSGAAPLGKELEDAVRAKFPNAKLGQGYGMTEAGPVLAMCLAFAKEPFDIKSGACGTVVRNAEMKIVDPDTGCSLPRNQPGEICIRGDQIMKGYLNDPEATARTIEKEGWLHTGDIGYIDDDDELFIVDRLKELIKYKGFQVAPAELEALLVNHPNISDAAVVPMKDEQAGEVPVAFVVRSNGSTITEDEVKDFISKQVIFYKRIKRVFFVETVPKSPSGKILRKDLRARLAAGISNCSRQAPFPLSISCN

SEQ ID NO: 11 - DNA, 1638 bp, Solanum lycopersicum
  mutation (720)..(722): mutation of SEQ ID NO1 Gln274His
atgccgatggataccgaaacaaatcaatcaggagatttcatctttcgatctaagctccctgatatttacatccctaaacatttgcctttacattcttattgctttgaaaatctttcggagttcagttctcgcccctgtttgattgatggttcaaatgatcgaatctacacttatgctgaagttgaacttacttcgagaaaagttgctgttgggcttagcaaattagggatccaacagaaggatacaatcatgatcctcttgcctaattgccctgagtttgtgtttgcgtttattggagcatcgtatctaggagcaatttcaacaatggctaatcctctgtttacaccagcagaggtagtaaaacaagccaaagcctcaagcgctaagatcgttatcacacaagcttgttttgcggggaaagtgaaggactacgcaattgaaaacgatttgaaggttatttgcgtcgactccgcaccggaaggttgtgttcatttctccgaattgattcaatccgatgaacacgaaattcctgaggtgaatatccagccgaacgatgtcgtagctctgccgtattcctccggtactaccgggctgccgaaaggggtgatgttgacacacaaaggcttagtcacgagcgttgcgcaacaagttgatggagaaaatgctaatttgtatatgcacagtgatgatgtgttgatgtgtgtgttgcctctgtcccatatatactcactcaattctgttttgctctgtgcattgagagtcggagcagcaattttgattatgcagaaattcgacattgctcagtttttggagttgatacagaagcataaagtgacgattgggccatttgtaccgcctattgttctcgccattgctaagagcccattagttgataactacgatctttcatcagtaagaacagtcatgtctggtgctgcaccattaggcaaggaacttgaagatgccgtccgagccaaattccctaacgctaaactcggtcagggttatggaatgacggaagctggtccagtgctggcgatgtgtttggcatttgcgaaagaaccgtttgatattaaatcaggggcatgtggtaccgttgtgaggaacgcagagatgaaaattgtggatccggatacgggttgctctctgccccgtaaccaacccggtgaaatttgcattagaggtgatcaaatcatgaaaggttacttgaatgaccctgaagctacagctagaacaatagaaaaagaaggatggttacacactggcgatattggatatattgacgatgatgatgagcttttcatcgtggatcgattgaaggaattgatcaaatacaaaggatttcaagtggcgcctgctgaactcgaagcacttcttgtcaaccaccctaacatttctgatgctgctgttgtcccaatgaaagatgaacaagcgggagaagttccagtggcttttgttgttagatcaaatggatctaccattactgaggatgaagtgaaggatttcatctccaagcaggtgatattctataagagaataaagcgtgtatttttcgtggagacggtaccgaaatctccatcaggaaaaattctgagaaaagacttaagagctagactggctgctggtatttcaaattga

SEQ ID NO: 12 - protein, 559 aa, Solanum lycopersicum
  VARIANT (274)..(274): variant of SEQ ID NO2 Gln274His
MPMDTETNQSGDFIFRSKLPDIYIPKHLPLHSYCFENLSEFSSRPCLIDGSNDRIYTYAEVELTSRKVAVGLSKLGIQQKDTIMILLPNCPEFVFAFIGASYLGAISTMANPLFTPAEVVKQAKASSAKIVITQACFAGKVKDYAIENDLKVICVDSAPEGCVHFSELIQSDEHEIPEVNIQPNDVVALPYSSGTTGLPKGVMLTHKGLVTSVAQQVDGENANLYMHSDDVLMCVLPLFHIYSLNSVLLCALRVGAAILIMQKFDIAQFLELIHKHKVTIGPFVPPIVLAIAKSPLVDNYDLSSVRTVMSGAAPLGKELEDAVRAKFPNAKLGQGYGMTEAGPVLAMCLAFAKEPFDIKSGACGTVVRNAEMKIVDPDTGCSLPRNQPGEICIRGDQIMKGYLNDPEATARTIEKEGWLHTGDIGYIDDDDELFIVDRLKELIKYKGFQVAPAELEALLVNHPNISDAAVVPMKDEQAGEVPVAFVVRSNGSTITEDEVKDFISKQVIFYKRIKRVFFVETVPKSPSGKILRKDLRARLAAGISNCSRQAPFPLSISCN

SEQ ID NO: 13 - DNA, 1681 bp, Solanum lycopersicum
  mutation (805)..(807): mutation of SEQ ID NO1 Phe269Leu
atgccgatggataccgaaacaaatcaatcaggagatttcatctttcgatctaagctccctgatatttacatccctaaacatttgcctttacattcttattgctttgaaaatctttcggagttcagttctcgcccctgtttgattgatggttcaaatgatcgaatctacacttatgctgaagttgaacttacttcgagaaaagttgctgttgggcttagcaaattagggatccaacagaaggatacaatcatgatcctcttgcctaattgccctgagtttgtgtttgcgtttattggagcatcgtatctaggagcaatttcaacaatggctaatcctctgtttacaccagcagaggtagtaaaacaagccaaagcctcaagcgctaagatcgttatcacacaagcttgttttgcggggaaagtgaaggactacgcaattgaaaacgatttgaaggttatttgcgtcgactccgcaccggaaggttgtgttcatttctccgaattgattcaatccgatgaacacgaaattcctgaggtgaatatccagccgaacgatgtcgtagctctgccgtattcctccggtactaccgggctgccgaaaggggtgatgttgacacacaaaggcttagtcacgagcgttgcgcaacaagttgatggagaaaatgctaatttgtatatgcacagtgatgatgtgttgatgtgtgtgttgcctctgttccatatatactcactcaattctgttttgctctgtgcattgagagtcggagcagcaattttgattatgcagaaattcgacattgctcagcttttggagttgatacagaagcataaagtgacgattgggccatttgtaccgcctattgttctcgccattgctaagagcccattagttgataactacgatctttcatcagtaagaacagtcatgtctggtgctgcaccattaggcaaggaacttgaagatgccgtccgagccaaattccctaacgctaaactcggtcagggttatggaatgacggaagctggtccagtgctggcgatgtgtttggcatttgcgaaagaaccgtttgatattaaatcaggggcatgtggtaccgttgtgaggaacgcagagatgaaaattgtggatccggatacgggttgctctctgccccgtaaccaacccggtgaaatttgcattagaggtgatcaaatcatgaaaggttacttgaatgaccctgaagctacagctagaacaatagaaaaagaaggatggttacacactggcgatattggatatattgacgatgatgatgagcttttcatcgtggatcgattgaaggaattgatcaaatacaaaggatttcaagtggcgcctgctgaactcgaagcacttcttgtcaaccaccctaacatttctgatgctgctgttgtcccaatgaaagatgaacaagcgggagaagttccagtggcttttgttgttagatcaaatggatctaccattactgaggatgaagtgaaggatttcatctccaagcaggtgatattctataagagaataaagcgtgtatttttcgtggagacggtaccgaaatctccatcaggaaaaattctgagaaaagacttaagagctagactggctgctggtatttcaaattgactcgagacaggccccttttcctttgtcgatatcatgtaattag

SEQ ID NO: 14 - protein, 559 aa, Solanum lycopersicum
  VARIANT (269)..(269): variant of SEQ ID NO2 Phe269Leu
MPMDTETNQSGDFIFRSKLPDIYIPKHLPLHSYCFENLSEFSSRPCLIDGSNDRIYTYAEVELTSRKVAVGLSKLGIQQKDTIMILLPNCPEFVFAFIGASYLGAISTMANPLFTPAEVVKQAKASSAKIVITQACFAGKVKDYAIENDLKVICVDSAPEGCVHFSELIQSDEHEIPEVNIQPNDVVALPYSSGTTGLPKGVMLTHKGLVTSVAQQVDGENANLYMHSDDVLMCVLPLFHIYSLNSVLLCALRVGAAILIMQKFDIAQLLELIQKHKVTIGPFVPPIVLAIAKSPLVDNYDLSSVRTVMSGAAPLGKELEDAVRAKFPNAKLGQGYGMTEAGPVLAMCLAFAKEPFDIKSGACGTVVRNAEMKIVDPDTGCSLPRNQPGEICIRGDQIMKGYLNDPEATARTIEKEGWLHTGDIGYIDDDDELFIVDRLKELIKYKGFQVAPAELEALLVNHPNISDAAVVPMKDEQAGEVPVAFVVRSNGSTITEDEVKDFISKQVIFYKRIKRVFFVETVPKSPSGKILRKDLRARLAAGISNCSRQAPFPLSISCN

SEQ ID NO: 15 - DNA, 1681 bp, Solanum lycopersicum
  mutation (556)..(558): mutation of SEQ ID NO1 Val186ILE
atgccgatggataccgaaacaaatcaatcaggagatttcatctttcgatctaagctccctgatatttacatccctaaacatttgcctttacattcttattgctttgaaaatctttcggagttcagttctcgcccctgtttgattgatggttcaaatgatcgaatctacacttatgctgaagttgaacttacttcgagaaaagttgctgttgggcttagcaaattagggatccaacagaaggatacaatcatgatcctcttgcctaattgccctgagtttgtgtttgcgtttattggagcatcgtatctaggagcaatttcaacaatggctaatcctctgtttacaccagcagaggtagtaaaacaagccaaagcctcaagcgctaagatcgttatcacacaagcttgttttgcggggaaagtgaaggactacgcaattgaaaacgatttgaaggttatttgcgtcgactccgcaccggaaggttgtgttcatttctccgaattgattcaatccgatgaacacgaaattcctgaggtgaatatccagccgaacgatatcttagctctgccgtattcctccggtactaccgggctgccgaaaggggtgatgttgacacacaaaggcttagtcacgagcgttgcgcaacaagttgatggagaaaatgctaatttgtatatgcacagtgatgatgtgttgatgtgtgtgttgcctctgttccatatatactcactcaattctgttttgctctgtgcattgagagtcggagcagcaattttgattatgcagaaattcgacattgctcagtttttggagttgatacagaagcataaagtgacgattgggccatttgtaccgcctattgttctcgccattgctaagagcccattagttgataactacgatctttcatcagtaagaacagtcatgtctggtgctgcaccattaggcaaggaacttgaagatgccgtccgagccaaattccctaacgctaaactcggtcagggttatggaatgacggaagctggtccagtgctggcgatgtgtttggcatttgcgaaagaaccgtttgatattaaatcaggggcatgtggtaccgttgtgaggaacgcagagatgaaaattgtggatccggatacgggttgctctctgccccgtaaccaacccggtgaaatttgcattagaggtgatcaaatcatgaaaggttacttgaatgaccctgaagctacagctagaacaatagaaaaagaaggatggttacacactggcgatattggatatattgacgatgatgatgagcttttcatcgtggatcgattgaaggaattgatcaaatacaaaggatttcaagtggcgcctgctgaactcgaagcacttcttgtcaaccaccctaacatttctgatgctgctgttgtcccaatgaaagatgaacaagcgggagaagttccagtggcttttgttgttagatcaaatggatctaccattactgaggatgaagtgaaggatttcatctccaagcaggtgatattctataagagaataaagcgtgtatttttcgtggagacggtaccgaaatctccatcaggaaaaattctgagaaaagacttaagagctagactggctgctggtatttcaaattgactcgagacaggccccttttcctttgtcgatatcatgtaattag

SEQ ID NO: 16 - protein, 559 aa, Solanum lycopersicum
  VARIANT (186)..(186): variant of SEQ ID NO2 Val186 ILE
MPMDTETNQSGDFIFRSKLPDIYIPKHLPLHSYCFENLSEFSSRPCLIDGSNDRIYTYAEVELTSRKVAVGLSKLGIQQKDTIMILLPNCPEFVFAFIGASYLGAISTMANPLFTPAEVVKQAKASSAKIVITQACFAGKVKDYAIENDLKVICVDSAPEGCVHFSELIQSDEHEIPEVNIQPNDILALPYSSGTTGLPKGVMLTHKGLVTSVAQQVDGENANLYMHSDDVLMCVLPLFHIYSLNSVLLCALRVGAAILIMQKFDIAQFLELIQKHKVTIGPFVPPIVLAIAKSPLVDNYDLSSVRTVMSGAAPLGKELEDAVRAKFPNAKLGQGYGMTEAGPVLAMCLAFAKEPFDIKSGACGTVVRNAEMKIVDPDTGCSLPRNQPGEICIRGDQIMKGYLNDPEATARTIEKEGWLHTGDIGYIDDDDELFIVDRLKELIKYKGFQVAPAELEALLVNHPNISDAAVVPMKDEQAGEVPVAFVVRSNGSTITEDEVKDFISKQVIFYKRIKRVFFVETVPKSPSGKILRKDLRARLAAGISNCSRQAPFPLSISCN

SEQ ID NO: 17 - DNA, 31 bp, Artificial Sequence
  synthetic primer
gaattcatgccgatggataccgaaacaaatc

SEQ ID NO: 18 - DNA, 26 bp, Artificial Sequence
  synthetic primer
ctcgagttaattagaatttggaaatg

SEQ ID NO: 19 - DNA, 29 bp, Artificial Sequence
  synthetic primer
gaattcatggtcaccgtggaggagtatcg

SEQ ID NO: 20 - DNA, 28 bp, Artificial Sequence
  synthetic primer
ctcgagctaagcagcaacactgtgaagg

(19) United States
(12) Patent Application Publication
(10) Pub. No.: US 2014/0033370 A1
(43) Pub. Date: Jan. 30, 2014

(54) VISUAL SCREENING FOR ENZYMES ON THE PHENYLPROPANOID PATHWAY

(71) Applicants: Hagai Abeliovich, Rehovot (IL); Moti Alberstein, Tel Aviv (IL)

(72) Inventors: Hagai Abeliovich, Rehovot (IL); Moti Alberstein, Tel Aviv (IL)

(73) Assignee: Yissuem Research Development Company of the Hebrew University of Jerusalem LTD., Jerusalem (IL)

(21) Appl. No.: 13/992,742

(22) PCT Filed: Dec. 8, 2011
(86) PCT No.: PCT/IL11/50055

§ 371 Date: Jun. 9, 2013

Related U.S. Application Data
(60) Provisional application No. 61/421,312, filed on Dec. 9, 2010.

(51) Int. Cl. C12Q 1/68; C12N 9/88; C12Q 1/25; C12N 9/00; C12Q 1/527; C12Q 1/26

(52) U.S. Cl. CPC C12Q 1/6897; C12Q 1/527; C12Q 1/26; C12Q 1/25; C12N 9/93; C12N 9/88; C12N 9/0073
USPC 800/298; 435/6.18; 506/11; 536/23.2; 435/419; 435/29; 435/25

(57) ABSTRACT

The invention relates to a method to screen for a nucleic acid molecule encoding a polypeptide involved in the synthesis of naringenin chalcone which demonstrates altered expression, activity or substrate specificity. The invention further relates to nucleic acid molecules identified by said screen and to transgenic plants modified to express said nucleic acid molecule.

### RELATED APPLICATIONS

The present application gains priority from U.S. Provisional Patent Application No. 61/421,312 filed Dec. 9, 2010 and which is included by reference as if fully set forth herein.

### BACKGROUND OF THE INVENTION

The phenylpropanoid pathway is a major, plant specific metabolic pathway that plays an important role in determining quality of plant-derived products. The distribution of flux through the phenylpropanoid pathway regulates the relative investment made by the plant in synthesizing lignin, suberin, flavonoids, isoflavonoids, stilbenoids, aurones and volatile phenylpropanoids. In determining fruit aroma and quality, levels of volatile phenylpropanoids are of central importance, while the levels of flavonoids, anthocyanins and stilbenoids impact both the nutritional value as well as taste characteristics. Thus, an important goal in plant breeding is the manipulation of the levels of various phenylpropanoids.

### SUMMARY OF THE INVENTION

The present inventors have developed two molecular evolution platforms designed for high throughput screening of hyperactive variants of early phenylpropanoid pathway enzymes. These platforms are based on the fact that yeast colonies that synthesize naringenin chalcone are coloured yellow in contrast with normal cream-coloured colonies. This allows for the screening for stronger-coloured colonies that produce higher levels of phenylpropanoids, or for colonies with changes in colour that indicate changes in substrate specificity or reaction trajectory.

By combining this selection with random mutagenesis of one of four enzymes, genetic variants with higher activity or novel genes encoding enzymes with specific catalytic properties can be selected. The present inventors propose that variants identified in this fashion can be used in metabolic engineering applications, to increase yield, as well as by reintroduction of the variants into plants to obtain plant material with altered metabolic profiles.

According to a first aspect of the invention, there is provided a method to screen for a nucleic acid molecule encoding a polypeptide involved in the synthesis of naringenin chalcone the method comprising:

(a) providing a yeast transformed with a test nucleic acid molecule encoding a polypeptide involved in the production of naringenin chalcone;

(b) culturing the yeast in conditions which allow the production of naringenin chalcone wherein the production of naringenin chalcone is associated with the production of a colour;

(c) comparing the colour produced by the yeast transformed with the test nucleic acid molecule with the colour produced by a control yeast (which was not transformed with the test nucleic acid molecule).

A difference in the colour, for example in terms of the intensity of the colour, generally a yellow colour, produced by the yeast transformed with the test polypeptide and the colour produced by the control yeast indicates that the test nucleic acid molecule shows altered expression, activity and/or substrate specificity. Thus the method of the invention may further comprise the step of selecting for a test nucleic acid molecule which, when transformed into yeast, produces a colour of intensity greater than that produced by the control yeast. The colour intensity can be measured by eye or can be measured spectrometrically.

The test nucleic acid molecule screened in accordance with the present invention may be a nucleic acid variant of a wild type nucleic acid molecule which encodes polypeptides involved in the production of naringenin chalcone. The polypeptide encoded by the nucleic acid variant may differ from polypeptide encoded by the wild type nucleic acid molecule by the addition, deletion or substitution of at least one amino acid residue. Thus the method of the invention may further comprise the step of determining the nucleic acid sequence of the test nucleic acid molecule.

The test nucleic acid molecule may encode a polypeptide, or polypeptide variant thereof, selected from the group consisting of phenylalanine ammonia lyase (PAL) (as represented in FIG. 1), coumarate 4-hydroxylase (C4H) (as represented in FIG. 2), 4-coumarate ligase (4CL) (as represented in FIG. 3), and chalcone synthase (CHS) (as represented in FIG. 4).

In a preferred method of the invention, there is provided:

(a) mutagenising a nucleic acid sequence wherein the nucleic acid sequence encodes a polypeptide involved in the production of naringenin chalcone to provide a test nucleic acid molecule comprising the mutagenised nucleic acid sequence;

(b) providing a yeast transformed with said test nucleic acid molecule encoding a polypeptide involved in the production of naringenin chalcone;

(c) culturing the yeast in conditions which allow the production of naringenin chalcone wherein the production of naringenin chalcone is associated with the production of a colour;

(d) comparing the colour produced by the yeast transformed with the test nucleic acid molecule with the colour produced by a control yeast (which was not transformed with the test nucleic acid molecule).

In a preferred method of the invention, the yeast are transformed with nucleic acid molecules encoding those polypeptides required for the synthesis of naringenin chalcone wherein the test nucleic acid molecules replaces the corresponding wild type nucleic acid molecule. The yeast may be transformed with nucleic acid molecules encoding each of the polypeptides involved in the synthesis of naringenin chalcone apart from the wild type equivalent of the test nucleic acid molecule wherein the test nucleic acid molecule replaces the corresponding wild type nucleic acid molecule. The yeast may be cultured in the presence of those substrates required for the production of naringenin chalcone.

In accordance with one aspect, the present invention concerns a method for high throughput screening for an enzyme in the early phenylpropanoid pathway with desired activity, the method comprising:
a) transforming a plurality of yeasts with a plurality of sequences coding for different versions of the candidate enzyme to be screened, said enzyme, optionally in the presence if additional enzymes in the early phenylpropanoid pathway, being capable of producing naringenin chalcone having a yellow colour;
b) providing said yeast in a manner where each different version of the candidate enzyme can be assigned to a specific yeast colony or cell,
c) providing conditions enabling enzymatic activity in the phenylpropanoid pathway for the production of naringenin chalcone,
d) comparing the yellow colour produced by the plurality of yeast colonies to control,
e) selecting those yeast colonies showing high yellow colour or different colour as compared to control,
f) determining the sequence of the variants of the candidate enzyme in the selected yeast, thereby identifying an enzyme with the required catalytic activity.

The aim of the invention is to select for enzymes in the early phenylpropanoid pathway with desired activity. One possibility is to increase the activity as compared to a control, which can be for example a WT version of the enzyme. Other potential aspects for selection are changes in substrate specificity, reaction mechanism, and increasing the desired activity under unique conditions such as stress conditions, presence of inhibitors, low levels of substrates etc

The enzymes to be screened are one of more of phenylalanine ammonia lyase (PAL), coumarate 4-hydroxylase (C4H), 4-coumarate ligase (4CL) and chalcone synthase (CHS) which are represented in FIG. 11 showing their position in the pathway.

In accordance with the aspect mentioned in the steps above not ALL the enzymes of the early phenylpropanoid pathway are necessarily expressed for a screening procedure, but rather only the minimal enzymes that are needed for production of naringenin chalcone. In fact this means that that what is needed is the sequences coding for the enzyme to be screened (present in different screenable versions), as well as additional downstream or upstream enzymes minimally required for naringenin chalcone production. The initial substrate depends on the first enzyme in the chosen expression scheme.

The first step of the method is transforming the yeast with sequences of the “supporting genes” encoding for enzymes that will not be screened but are required for generating naringenin chalcone together with the gene to be screened, for example if 4CL variants are screened, one will also introduce CHS. Transformation of the yeasts can be done as previously described {Gietz and Woods, 2002, Methods Enzymol, 350, 87-96}. Preferentially, integrating plasmids (e.g. pRS30X series) should be used for introducing the “supporting genes”.

The yeast are then transformed with sequences coding for different versions of the candidate enzyme, the sequences differ from each other in the coding sequence. The different versions may be obtained by mutagenesis (either random or directed), or may be naturally occurring homologous or orthologous sequences found in various species or varieties of plants, or may be an expression library made from a specific plant, plant tissue, or other organism.

The enzyme screened should be transformed into yeast, optionally together with other enzymes which are not screened that are required for the production of naringenin chalcone (the “supporting genes”). For example the screened enzyme may be CHS, and in that case there is no need to provide additional enzymes, as in the presence of its substrates coumaroyl-coA, it can produce the naringenin chalcone.

Conceivably one could try to screen by introducing CHS without previously introducing 4CL, and use coumaroyl-CoA as an externally provided substrate. However coumaroyl-CoA does not readily enter cells, is relatively unstable in aqueous solution, and is expensive. It is therefore preferable, even when screening for versions of CHS, to use yeast that already express 4CL and to use coumaric acid as substrate.

Of course if desired to screen for versions of 4CL with increased activity, one can transform the yeast with sequences coding for different candidate 4CL versions, assuming the yeast were previously transformed with a plasmid expressing the “supporting gene” CHS, and to provide coumarate as a substrate and test for activity.

To test or select for C4H activity, one initially transforms the yeast either with 4CL and CHS (in which case cinnamic acid needs to be added as substrate) or with PAL, 4CL, and CHS (in which case no exogenous substrate is needed). One then transforms the yeast with different versions of C4H coding sequences and tests the transformants for evolution of yellow colour either with or without cinnamic acid as substrate (depending on the system used).

If it is desired to go further upstream and screen for the first enzyme in the pathway PAL, then the yeast can be initially be transformed with C4H, 4CL, and CHS, and then with different versions of sequences coding for candidate versions of PAL, as well as (non-candidate, supporting) versions of C4H, 4CL and CHS. In this case there is no need to provide externally with the substrate of PAL, phenylalanine, as it naturally occurs in yeast.

The following table 1 summarizes these options

| | Additional | | |
|---|---|---|---|
| Enzyme | enzymes required | | |
| to be | for production of | External | |
| screened | colour | substrate | Comments |
| CHS | none | coumaroyl-coA | Difficulty in providing |
| | | | these substrates |
| CHS | 4CL | Coumarate | This is the substrate for |
| | | | 4CL |
| 4CL | CHS | Coumarate | |
| PAL | C4H and CHS | None | The substrate of PAL |
| | and 4CL | | phenylalanine naturally |
| | | | occurs in yeast. Can be |
| | | | enhanced by |
| | | | overexpressing CPR. |

Another alternative for screening, that is preferable in accordance with the invention is to transform the yeast with all the sequences required for the complete early phenylpropanoid pathway wherein all the sequences of the other enzymes, save for the sequence of the enzyme to be selected, are identical in all yeast cells/colonies, and only sequences of the screened enzyme has different versions in the different cells/colonies.

The first step of the method is transforming the yeast with sequences coding for all the enzymes necessary for generating naringenin chalcone from phenylalanine or from an exogenous substrate, except the gene to be selected. For example in the case of 4CL, one can transform with PAL, C4H, and CHS. The second step is to generate variants of the gene to be selected (if one is screening for changes in activity) or an expression library that presumably contains the gene or genes encoding enzymes with the desired property.

In accordance with a further aspect of the present invention there is provided a method for high throughput screening for an enzyme in the early phenylpropanoid pathway with desired activity, the method comprising:
a) generating a yeast strain containing all the genes required for production of naringenin chalcone, minus the gene to be screened;
b) transforming the plurality of above yeasts with a plurality of sequences coding for different versions of the candidate enzyme to be screened;
c) providing said yeast in a manner where each different version of the candidate enzyme can be assigned to a specific yeast colony or cell,
d) providing conditions enabling enzymatic activity in the phenylpropanoid pathway for the production of naringenin chalcone,
e) comparing the yellow colour produced by the plurality of yeast colonies to control,
f) selecting those yeast colonies showing high yellow colour as compared to control,
g) determining the sequence of the variants of the candidate enzyme in the selected yeast, thereby identifying an enzyme with the required catalytic activity.

The yeast are grown under conditions (temperatures, nutrients, substrates) that enable enzymatic activity and activity is detected by appearance and strength of yellow colour.

Once candidate colonies are picked, either in accordance with the first or the second options, they are re-streaked and the colour and its intensity are compared to control. For example the control would be yeast transformed under the same conditions with a wild type version of the enzymes, and clones featuring higher yellow colour relative to the control are selected. As the sequence coding for the candidate enzyme is known in the selected yeast, it is possible to have a sequence for a hyperactive enzyme variant.

The selected sequence can be used either in production of desired materials in host cells or alternatively, the selected sequence can be used to replace the endogenous plant gene in order to generate plants with altered metabolic profiles. A third application is to use the sequence information in a TILLING screen {Barkley and Wang, 2008, Curr Genomics, 9, 212-26} for identical or similar natural or induced mutations in plants.

By using the method of the present invention the inventors were able to select for six hyper active 4CL mutants (obtained by random mutagenesis and selected by the method of the invention). A table of the presently available mutants and their points of variation with wild-type 4CL is shown below.

| Mutant | position | from | to | comments |
|---|---|---|---|---|
| 12_1 | 235 | Val | Met | |
| 12_1 | 237 | Pro | Pro | Silent mutation |
| 12_1 | 325 | Ala | Gly | |
| 18-1 | 239 | Phe | Ser | |
| 21_1 | 224 | Leu | Leu | Silent mutation |
| 21_1 | 274 | Gln | His | |
| 2 | 269 | Phe | Leu | |
| 11-1-3 | 186 | Val | G | |
| 1 | 186 | Val | Ile | |
| 1 | 187 | Val | Leu | |

In a further aspect of the invention there is provided a nucleic acid molecule identified by the method of the present invention.

The nucleic acid molecule identified by the method of the present invention may be represented by the sequence shown in any one of FIGS. 5-10 (a). Thus the present invention further provides a nucleic acid molecule as represented by the sequence shown in any one of FIGS. 5-10 (a) and which has 4CL enzyme activity.

In a further aspect of the invention there is provided a polypeptide having 4CL enzyme activity wherein the polypeptide is represented by the sequence shown in any one of FIGS. 5-10(b).

The invention further provides a vector adapted for expression of a nucleic acid molecule according to the invention. The vector may be provided with a promoter that enables the expression of said nucleic acid molecule to be regulated.

The invention further provides a host cell transformed with the vector of the invention.

The invention further provides a transgenic plant transformed with a nucleic acid molecule comprising a nucleic acid sequence operably linked to a promoter, said nucleic molecule being identified by a method of the invention. Preferably, the transgenic plant is transformed with a nucleic acid sequence as represented by the sequence shown in any one of FIGS. 5-10 (a). The promoter may be a tissue specific promoter for example an epidermal specific promoter. The transgenic plant may be modified such that the transformed nucleic acid molecule replaces the endogenous wild type nucleic acid molecule in the plant.

The invention further provides a seed produced by a transgenic plant according to the invention.

The invention further provides a transgenic plant cell comprising a nucleic acid molecule identified by the method of the present invention, for example a nucleic acid molecule as represented by the sequence shown in any one of FIGS. 5-10 (a).

The invention further provides a method to generate a plant with improved nutritional value and or taste comprising:

i) providing a plant cell or seed according to the invention;

ii) regenerating said cell or seed into a plant; and optionally

iii) determining the nutritional value and/or organoleptic properties of said plant.

If desired it is possible to use codons which are suitable for the organism where the sequences are to be expressed.

The present invention further concerns expression constructs comprising any one of the nucleic acid sequences mutants 12-1, 11-1-3, 18-1, 21-1, 2 and 1.

The present invention further concerns host cells transformed by the sequences of the nucleic acid sequences of mutants 12-1, 11-1-3, 18-1, 21-1, 2 and 1, and transgenic or nontransgenic plants expressing the amino acid sequences of the mutants 12-1, 11-13, 18-1, 21-1, 2 and 1

A further aspect of the invention provides a method to screen for agents which modulate the activity of one or more polypeptides involved in the synthesis of naringenin chalcone comprising the steps of:

(a) providing a yeast transformed with nucleic acid molecules encoding polypeptides involved in the production of naringenin chalcone;

(b) culturing the transformed yeast under conditions which allow the production of naringenin chalcone, in the presence of at least one agent to be tested, wherein the production of naringenin chalcone is associated with the production of a colour;

(c) comparing the colour produced by the yeast cultured in the presence of the agent to be tested with the colour produced by a control yeast (which was cultured in the absence of at least one agent to be tested).

Preferably the yeast are transformed with nucleic acid molecules encoding some or all of phenylalanine ammonia lyase, coumarate 4-hydroxylase, 4-coumarate ligase and chalcone synthase, under the control of the CUP1 promoter. The transformed yeast cells will generate naringenin chalcone from endogenous phenylalanine in the presence of sum-micromolar concentrations of copper ions.

A difference in the colour, for example in terms of the intensity of the colour, generally a yellow colour, produced by the yeast cultured in the presence of the agent to be tested and the colour produced by the control yeast indicates that agent has modulated the activity of one or more of the polypeptides involved in the synthesis of naringenin chalcone.

The agent to be tested may be an inhibitor or activator of an enzyme involved in the synthesis of naringenin chalcone.

A further aspect of the invention provides a method to screen for proteins, in particular plant proteins, which modulate the activity of one or more polypeptides involved in the synthesis of naringenin chalcone comprising the steps of:

a) providing a yeast transformed with nucleic acid molecules encoding polypeptides involved in the production of naringenin chalcone;

(b) transforming the yeast produced in (a) with one or more nucleic acid molecules encoding a protein that is naturally expressed in plants;

(c) culturing the transformed yeast from (b) under conditions which allow the production of naringenin chalcone wherein the production of naringenin chalcone is associated with the production of a colour;

(d) comparing the colour produced by the yeast cultured in (c) with the colour produced by the control yeast (which was not transformed with one or more nucleic acid molecules encoding a protein that is naturally expressed in plants).

Preferably the method comprises:

a) providing a yeast transformed with nucleic acid molecules encoding polypeptides involved in the production of naringenin chalcone;

(b) transforming the yeast produced in (a) with one or more nucleic acid molecules encoding a protein that is naturally expressed in plants;

(c) culturing the transformed yeast from (a) and from (b) under conditions which allow the production of naringenin chalcone wherein the production of naringenin chalcone is associated with the production of a colour;

(d) comparing the colour produced by the transformed yeast from (a) with the colour produced by the transformed yeast from (b).

Preferably the yeast are transformed with nucleic acid molecules encoding some or all of phenylalanine ammonia lyase, coumarate 4-hydroxylase, 4-coumarate ligase and chalcone synthase, under the control of the CUP1 promoter.

The present invention concerns a gene discovery platform for screening plant cDNA libraries for non-catalytic regulatory proteins that function in the modulation of the phenylpropanoid pathway. Such proteins, currently unknown, will be a major target for plant breeding strategies and genetic manipulation, with the aim of creating pre-designed changes in the output of the pathway. The method comprises:
a) transforming yeast with sequences encoding phenylalanine ammonia lyase, coumarate 4 hydroxylase, 4-coumarate ligase and chalcone synthase, under the control of the CUP1 promoter. These cells will generate naringenin chalcone from endogenous phenylalanine in the presence of sum-micromolar concentrations of copper ions.
b) Further transforming said yeast with gene libraries expressing plant genes.
c) Identifying colonies showing increased or decreased color, relative to control colonies transformed with empty vector.
d) determining the sequence of the variants of the candidate enzyme in the selected yeast, thereby identifying proteins that modulate the phenylpropanoid pathway.

A yet further aspect of the invention provides a yeast transformed with nucleic acid molecules encoding polypeptides involved in the production of naringenin chalcone, for example polypeptides having one or more the amino acid sequences represented in FIGS. 1 to 4 (b).

The invention further provides a kit suitable for screening for agents which modulate the activity of one or more polypeptides involved in the synthesis of naringenin chalcone comprising a yeast transformed with nucleic acid molecules encoding polypeptides involved in the production of naringenin chalcone.

A further aspect of the invention provides the use of a yeast or kit according to the invention in screening for agents, for example inhibitors, which modulate the activity of one or more polypeptides involved in the synthesis of naringenin chalcone

By another aspect the present invention concerns a method for screening for inhibitors of at least one gene in the early pehenypropanoid pathway.

By another aspect the present invention concerns a method for high throughput screening for an inhibitor or activator of at least one enzyme in the early phenylpropanoid pathway the method comprising:
a) transforming yeasts with sequences coding the enzymes of the early phenylpropanoid pathway which are capable, in the presence of suitable substrates, of producing naringenin chalcone having a yellow colour;
b) contacting said yeast with a plurality of candidates inhibitors in an array, such a manner that each different inhibitor can be assigned to a specific well or location in the array,
c) providing conditions enabling enzymatic activity in the phenylpropanoid pathway for the production of naringenin chalcone, comparing the yellow colour produced by the plurality of yeast colonies to control,
e) selecting those yeast colonies exhibiting low, and preferably no, yellow colour as compared to control, yet show no effects on cell growth.
f) determining the inhibitor that was contacted with the selected yeast thereby obtaining inhibitors of the enzyme.

Inhibitors for these enzymes may have various applications such as in herbicides, increasing/decreasing flavonoid content, increasing/decreasing lignin content, and increasing/decreasing volatile profiles in plants. In addition, as some human enzymes show similarity to these enzymes, this initial screening method may serve as a first step in the screening of drug candidate since it simultaneously selects for cell permeability and lack of general toxicity.

For example, 4CL is highly related to the ubiquitous acetyl-coA synthetases; PAL is closely related to histidine ammonia lyase, an enzyme that is involved in histidine catabolism and mutations of which lead to histidinemia, an autosomal recessive genetic disorder; C4H is a conserved member of the cytochrome P450 monooxygenase family.

Activators of these enzymes may also have various applications, such as in reversible plant growth modulation, color changes, and organoleptic properties.

A yeast strain is transformed with chromosomally-integrating constructs coding for the full early phenylproponoid pathway enzymes (PAL, CH4, 4CL and CHS). If the candidate compound is an inhibitor of one of the enzymes, (and the inhibitor can penetrate the cell), the yellow colour in the presence of the compound will be lower, or even non-existent as compared to the colour in the absence of the compound.

This method enables one to rapidly screen a large number of candidate compounds. Simultaneously, one will monitor cell density by measuring OD at 600 nanometers, to ensure that any change in yellow colour is not due to changes in cell growth or viability.

If it is desired later to pinpoint the exact enzyme which the inhibitor, selected by the above method, inhibits—it is always possible to test the inhibitor in a cell-free assay with each of the isolated enzymes.

In a further aspect, the invention provides a method to screen for and optionally measure copper contamination in a test sample comprising the steps of:

(a) providing a yeast transformed with a nucleic acid molecule comprising an expression cassette comprising the nucleic acid sequence as represented in FIGS. 3(a) and 4(a) operably linked to the CUPI promoter;

(b) culturing the transformed yeast in conditions which allow the production of naringenin chalcone, in the presence of a test sample suspected of containing copper, wherein the production of naringenin chalcone is associated with the production of a colour;

(c) comparing the colour produced by the yeast cultured in the presence of the test sample with the colour produced by a control yeast (which was cultured in the absence of the test sample).

Preferably the transformed yeast are cultured in the presence of the substrate, coumaric acid, and optionally submolecular levels of copper ion.

The sample may be any aqueous liquid sample for example a water sample.

A difference in the colour, for example in terms of the intensity of the colour, generally a yellow colour, produced by the yeast cultured in the presence of the test sample and the colour produced by the control yeast indicates that the test sample is contaminated with copper.

In one embodiment, the present invention concerns a method for screening for and measuring copper contaminants in water sources and the environment, the method comprising:

a) transforming yeasts with sequences coding 4-coumarate ligase and chalcone synthase under the control of the CUP1 promoter, which are capable, in the presence of coumaric acid and

sub-micromolar levels of copper ion, of producing naringenin chalcone, having a yellow colour;

b) contacting said yeast with 1 mM coumaric acid and with a test specimen of a water solution suspected of containing copper.

c) providing conditions enabling enzymatic activity in the phenylpropanoid pathway for the production of naringenin chalcone, and enhancing the colour output by alkalinization of the medium

d) comparing the yellow colour produced by the yeast culture to control lacking copper and a standard set of known copper concentrations

e) determining the copper concentration in the unknown sample by comparison with the standard curve.

Copper contamination is a growing concern for authorities in industrial and industrializing

nations and strict controls are enforced; The above method allows rapid visual output that can also be analyzed by either absorbance or fluorescence with robust S/N ratios, allowing both a rapid, cost effective visual approximation that requires no additional instrumentation, as well as a more stringent quantitative result utilizing a spectrophotometer or fluorimeter. The materials required are low-cost and non-toxic (other than an alkali base such as KOH). In addition the method is amenable to an instrument-free visual readout, that allows an estimate to be made in the field, in the absence of instrumentation (see FIG. 15).

Optional or preferred aspects or embodiments of the invention apply to each statement of the invention mutatis mutandis.

### BRIEF DESCRIPTION OF THE FIGURES

FIG. 1 discloses central pathways for phenylpropanoid biosynthesis

FIG. 2 discloses a colour generation by recombinant yeast producing naringenin chalcone. Cells expressing 4CL and CHS (right) or the appropriate empty vectors (left) were grown in liquid medium in the presence of 1 mM coumaric acid. The cells were pelleted by centrifugation and photographed. The container on the right shows a strong yellow colour and the container on the left shows only the normal cream colour.

FIG. 3 discloses an Isolation of hyperactive 4CL mutants.

A. HPLC chromatograms at 390 nm of yeast cell extracts containing an integrated CHS gene and either WT tomato 4CL gene or one of the 4CL mutants as mentioned in figures (11-1-3 and 18-1). Single peak representing naringenin chalcone is indicated (See materials and methods). B. Quantification of naringenin chalcone production relative to wild-type 4CL. Graph represents 2 independent experiments (error bars indicated).

FIG. 4 discloses In vitro kinetics of WT 4CL and hyperactive variants.

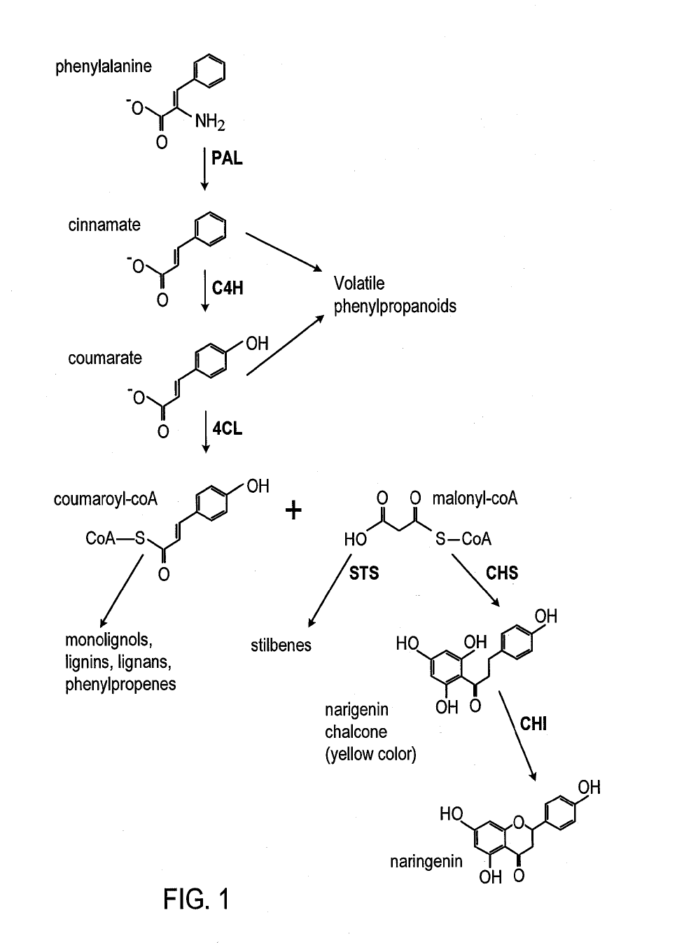

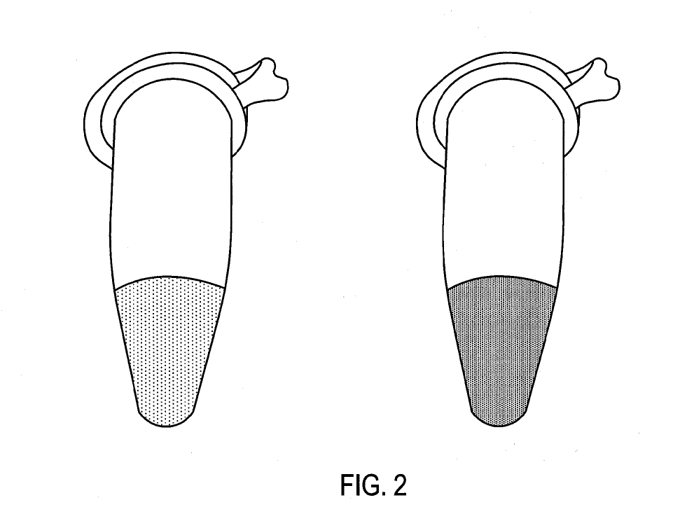

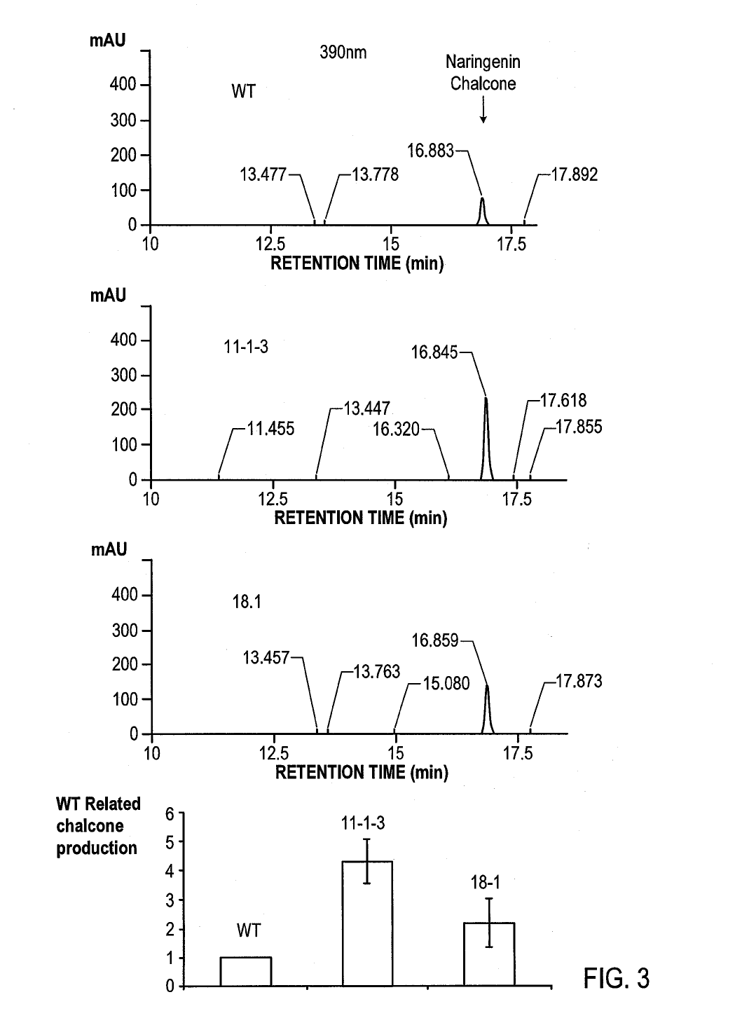

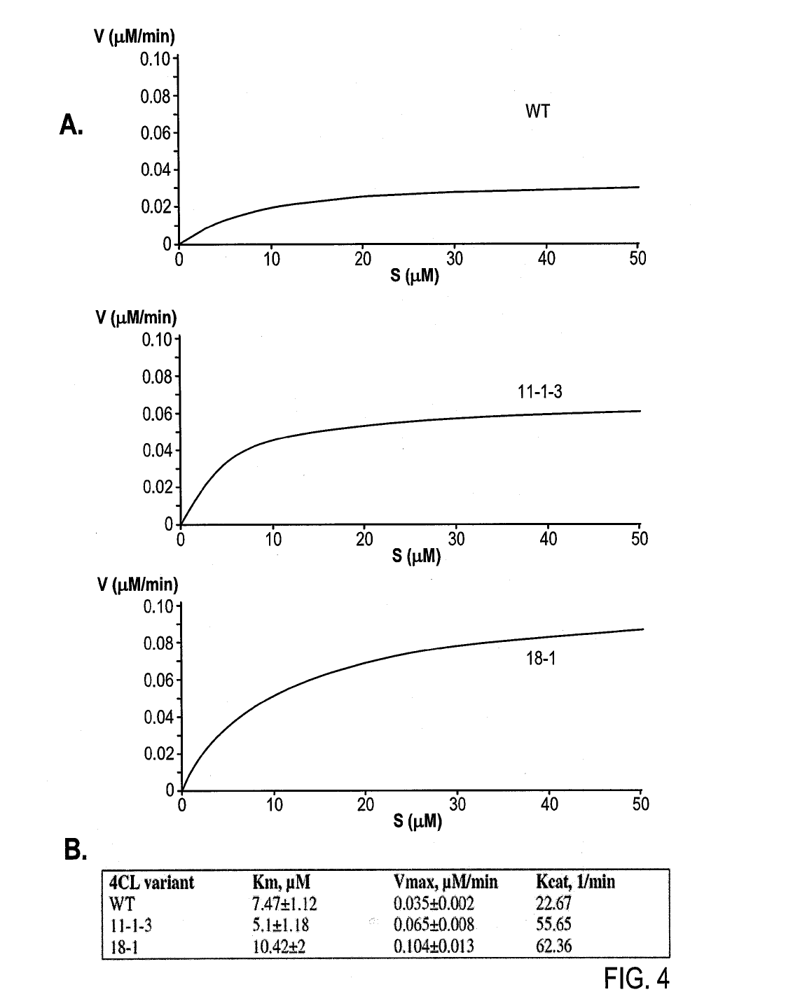

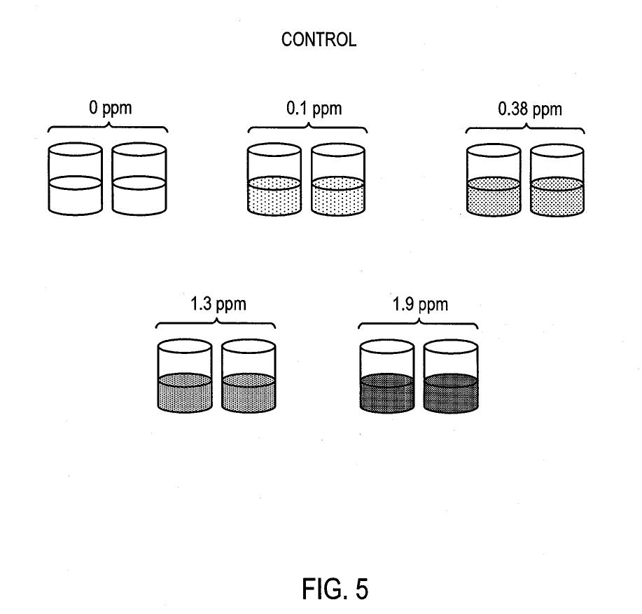

A. 4CL activity was measured spectrophotometrically with the microplate reader at 30° C. The 4CL reaction mixtures contained pure recombinant 4CL protein, ATP, MgSO4, CoA and elevated concentration of p-coumaric acid substrate (pH 7.8). Production of pcoumaroyl-CoA was measured at 333 nm (See materials and methods for further details). Kinetic graph represents of 4 independent experiments which were done in duplicate (statistic bar is also shown). The elucidated average kinetics values of 4CL tomato enzyme is shown at the bottom table. B. Quantitative analysis of the data in A. Curve fitting was according to Haines-Wolff and bars represent standard deviation

FIG. 5 A copper bioassay based on naringenin chalcone production in Saccharomyces cerevisiae. Yeast expressing 4CL and CHS genes were challenged with 1 mM coumaric and increasing Copper concentrations (0, 1.58 μM (0.1 ppm), 6 μM (0.38 ppm), 20.5 μM (1.3 ppm), 30 μM (1.9 ppm)) to produce naringenin chalcone. 48 hours after copper induction cells were visualized by adding 50% KOH solution (1:1 v).

### DETAILED DESCRIPTION OF EMBODIMENTS

Materials and methods

### Reagents

Reagents were from Sigma-Aldrich (Rehovot, Israel) unless otherwise specified.

DNA Isolation Plasmids Construction.

The full-length coding region of Solanum lycopersicon (S.l.) 4CL and CHS cDNA (amino acid residues 1-559 and 1-389 respectively) was amplified by PCR from total cDNA and was verified by sequencing. 4CL PCR fragment was digested with PstI and XhoI and subcloned into a pCu415 plasmid {Labbe and Thiele, 1999, Methods Enzymol, 306, 145-53}. The CHS PCR fragment was digested with EcoRI and XhoI and subcloned into pCu306 plasmid {Labbe and Thiele, 1999, Methods Enzymol, 306, 145-53} to generate yeast integrated pCu306[CHS] construct. pCu306 [CHS] was further digested with NcoI for genomic integration. For recombinant expression studies, 4CL was re-cloned into a pET28a expression vector (Novagen) using NdeI and NotI as the restriction sites.

Gene cloning and plasmid construction. A previously uncharacterized full length contig encoding a 4CL homolog in the Tomato Genome Database (solgenomics.net; currently accession number AK328438) was identified. The full length coding region of this contig (encoding residues 1-559) was amplified by PCR from tomato peel cDNA using the oligonucleotide primers 5′-GAATTCATGCCGATGGATACCGAAACAAATC-3′ and 5′-CTCGAGTTAATTAGAATTTGGAAATG-3′. A previously characterized tomato CHS (accession number X55194) was chosen and CHS cDNA (encoding amino acid residues 1-389) was amplified from the same tomato peel cDNA using the primers 5′-GAATTCATGGTCACCGTGGAGGAGTATCG-3′ and 5′-CTCGAGCTAAGCAGCAACACTGTGAAGG-3′ and was verified by sequencing. The 4CL PCR fragment was digested with PstI and XhoI and subcloned into the copper inducible expression vector pCu305 (Labbe and Thiele, 1999) to generate the pMAB1 plasmid. The CHS PCR fragment was digested with EcoRI and XhoI and subcloned into the copper-inducible pCu306 vector to generate the pMAB2 plasmid.

Yeast Strains.

Yeast cells containing an integrated CHS gene were transformed with either an empty vector, WT tomato 4CL gene or one of the 4CL mutants as mentioned in figures. Single colonies of the transformed yeast were grown overnight in a selective SD medium containing glucose as the carbon source and Bathocuproine disulphonate (BCS), a copper chelator at 30° C. in vigorous shaking till O.D.600 nm of 1.5. The next day cultures were diluted ×50 fold to an 0.03 O.D.600 nm into fresh 10 ml of selective SD medium containing p-coumaric (being the substrate of 4CL) acid a substrate and 30 μl CuSO4 to induced gene expression. Cultures were then incubated for further 8 days and cells were centrifuged 5 min 3500 RPM, supernatant were collected and pellets were washed with double distilled water. Cells extracts were obtained by vortexing the pellets for 30 min in the presence of 100 μl glass beads and 200 μl 70% methanol. Extracts were clarified by centrifugation for 10 min at 13,000×g.

### Yeast Growth Conditions

Yeast cells were grown in modified minimal medium (SD) (2% glucose, 6.7% yeast nitrogen base (Difco) without amino acid leucine) including 1 mM coumaric and increasing Copper concentrations (0, 1.58 μM (0.1 ppm), 6 μM (0.38 ppm), 20.5 μM (1.3 ppm), 30 μM (1.9 ppm).

HPLC Analysis.

Samples were analyzed by high performance liquid chromatography (HPLC) on an SB-C18 column (Agilent). The column was developed with a gradient profile of 10% solution B (acetonitrile), 90% solution A (1.5% (vol/vol) acetic acid in water) for 5 min, 40% B in 10 min, 60% B in 5 min and 95% B in 5 min. The Flow rate was 1 ml/min and the column was kept at 30° C. Naringenin and naringenin chalcone were quantified by the peak areas at 290 and 390 nm, respectively relative to naringenin standard (FLUKA) and chemically synthesized naringenin chalcone.

4CL Purification and In-Vitro Enzyme Kinetics Assay.

The pET28a-hisx6-4CL plasmid was transformed into E. coli BL21(DE3; Novagen) cells. Cells were grown overnight in LB medium supplemented with ampicillin and chloramphenicol, and diluted 1:10 for 1 h prior to induction with 1 mM IPTG. After 4 h of induction at 37° C., the cells were harvested by centrifugation, cell pellets were stored at −80° C. To generate extracts, cell pellets were then sonicated in lysis buffer (50 mM NaH2PO4, 300 mM NaCl, 10 mM imidazole, pH 8.0) on ice 5×30 sec and centrifuged 13000×g for 30 min at 4° C. Cell lysate was then equilibrated with 0.5 ml Ni-NTA beads (GIAGEN) and loaded on Disposable columns. The column was washed 5 times and eluted with increasing concentration of imidazole (30 mM to 80 mM). Eluted fractions eluted with increasing concentration of imidazole (30 mM to 80 mM). Eluted fractions samples were separated by SDS-PAGE and detected using Bromophenol Blue staining (left to right: 30 mM and then 50 mM fractions 1-5 and finally 80 mM). In-vitro 4CL activity was measured spectrophotometrically with the microplate reader at 30° C. The 4CL reaction mixtures contained pure recombinant 4CL protein, 5 mM ATP, 5 mM MgSO4, 5 mM CoA and varying concentration of p-coumaric acid substrate in 0.4 M Tris-HCl buffer (pH 7.8). Control reactions contained the same components without CoA. The reaction was initiated by the addition of ATP and incubated for 1 h at 30° C. Enzyme activity was measured as the increase in absorbance at the absorption maximum of p-coumaroyl-CoA at 333 nm. The extinction coefficient for p-coumaroyl-CoA (21 cm⁻¹ mM⁻¹, Lee et al. 1997) was used to calculate enzyme activity.

1. A visual/spectrophotometric method to screen for a nucleic acid molecule encoding a polypeptide involved in the synthesis of naringenin chalcone the method comprising:
(a) providing a yeast transformed with a test nucleic acid molecule encoding a polypeptide involved in the production of naringenin chalcone;
(b) culturing the yeast in conditions which allow the production of naringenin chalcone wherein the production of naringenin chalcone is associated with the production of a colour;
(c) comparing the colour produced by the yeast transformed with the test nucleic acid molecule with the colour produced by a control yeast (which was not transformed with the test nucleic acid molecule).

2. A method as claimed in claim 1 wherein a difference in the colour produced by the yeast transformed with the test polypeptide and the colour produced by the control yeast indicates that the test nucleic acid molecule shows altered expression, activity and/or substrate specificity.

3. A method as claimed in claim 1 further comprising the step of selecting for the yeast transformant which produces a colour of intensity greater than that produced by the control yeast.

4. A method as claimed in claim 3 further comprising isolating, and optionally sequencing, the test nucleic acid molecule from the selected yeast transformant.

5. A method as claimed in claim 1 wherein the test nucleic acid molecule encodes a polypeptide, or polypeptide variant thereof, selected from the group consisting of PAL, C4H, 4CL (as represented in SEQ ID NO:1 and 2), and CHS (as represented in SEQ ID NO:3 and 4).

6. A method as claimed in claim 1 comprising:
a) transforming a plurality of yeasts with a plurality of sequences coding for different versions of the candidate enzyme to be screened, said enzyme, optionally in the presence if additional enzymes in the early phenylpropanoid pathway, being capable of producing naringenin chalcone having a yellow colour;
b) providing said yeast in a manner where each different version of the candidate enzyme can be assigned to a specific yeast colony or cell,
c) providing conditions enabling enzymatic activity in the phenylpropanoid pathway for the production of naringenin chalcone,
d) comparing the yellow colour produced by the plurality of yeast colonies to control,
e) selecting those yeast colonies showing high yellow colour or different colour as compared to control,
f) determining the sequence of the variants of the candidate enzyme in the selected yeast, thereby identifying an enzyme with the required catalytic activity.

7-9. (canceled)

10. A method as claimed in claim 1 comprising:
a) generating a yeast strain containing all the genes required for production of naringenin chalcone, minus the gene to be screened;
b) transforming the plurality of above yeasts with a plurality of sequences coding for different versions of the candidate enzyme to be screened;
c) providing said yeast in a manner where each different version of the candidate enzyme can be assigned to a specific yeast colony or cell,
d) providing conditions enabling enzymatic activity in the phenylpropanoid pathway for the production of naringenin chalcone to a level that enables visual or spectrophotometric detection,
e) comparing the yellow colour produced by the plurality of yeast colonies to control,
f) selecting those yeast colonies showing high yellow colour as compared to control,
g) determining the sequence of the variants of the candidate enzyme in the selected yeast,
thereby identifying an enzyme with the required catalytic activity.

11. A nucleic acid molecule identified by the method of claim 1.

12. The nucleic acid as claimed in claim 11 wherein the nucleic acid molecule is represented by the sequence shown in any one of SEQ ID NOS: 5, 7, 9, 11, 13 or 15.

13. The nucleic acid molecule as claimed in claim 12 and which has 4CL enzyme activity.

14-16. (canceled)

17. A transgenic plant transformed with a nucleic acid molecule comprising a nucleic acid sequence operably linked to a promoter, said nucleic molecule being identified by a method as claimed in claim 1.

18. A plant as claimed in claim 17 wherein the plant is transformed with a nucleic acid sequence as represented by the sequence shown in any one of SEQ ID NOS: 5, 7, 9, 11, 13 or 15.

19. A seed produced by a transgenic plant as claimed in claim 17.

20. A transgenic plant cell comprising a nucleic acid molecule identified by a method as claimed in claim 1.

21. A cell as claimed in claim 20 wherein the plant is transformed with a nucleic acid sequence as represented by the sequence shown in any one of SEQ ID NOS:5, 7, 9, 11, 13 or 15.

22. (canceled)

23. A method to screen for agents which modulate the activity of one or more polypeptides involved in the synthesis of naringenin chalcone comprising the steps of:
(a) providing a yeast transformed with nucleic acid molecules encoding polypeptides involved in the production of naringenin chalcone;
(b) culturing the transformed yeast in conditions which allow the production of naringenin chalcone, in the presence of at least one agent to be tested, wherein the production of naringenin chalcone is associated with the production of a colour;
(c) comparing the colour produced by the yeast cultured in the presence of the agent to be tested with the colour produced by a control yeast (which was cultured in the absence of the at least one agent to be tested).

24. A method as claimed in claim 23 wherein the agent to be tested is an inhibitor of an enzyme involved in the synthesis of naringenin chalcone.

25. A method as claimed in claim 23 wherein the agent is a protein, in particular plant proteins, which modulate the activity of one or more polypeptides further comprising:
prior to (b) transforming the yeast produced in (a) with one or more nucleic acid molecules encoding a protein that is naturally expressed in plants,
(c) culturing the transformed yeast from (b) under conditions which allow the production of naringenin chalcone wherein the production of naringenin chalcone is associated with the production of a colour;
(d) comparing the colour produced by the yeast cultured in (c) with the colour produced by the control yeast (which was not transformed with one or more nucleic acid molecules encoding a protein that is naturally expressed in plants).

26. A method as claimed in claim 25 wherein the yeast are transformed in (a) with nucleic acid molecules encoding some or all of phenylalanine ammonia lyase, coumarate 4-hydroxylase, 4-coumarate ligase and chalcone synthase, optionally under the control of the CUP1 promoter.

27-31. (canceled)

32. A method to screen for, and optionally measure, copper contamination in a test sample comprising the steps of:
(a) providing a yeast transformed with a nucleic acid molecule comprising an expression cassette comprising the nucleic acid sequence as represented in SEQ ID NOS: 1 or 3 operably linked to the CUPI promoter;
(b) culturing the transformed yeast in conditions which allow the production of naringenin chalcone, in the presence of a test sample suspected of containing copper, wherein the production of naringenin chalcone is associated with the production of a colour;
(c) comparing the colour produced by the yeast cultured in the presence of the test sample with the colour produced by a control yeast (which was cultured in the absence of the test sample).
